(19) United States
(12) Patent Application Publication
(10) Pub. No.: US 2012/0165494 A1
(43) Pub. Date: Jun. 28, 2012

(54) METHODS FOR PRODUCING POLYOLS AND POLYURETHANES

(76) Inventors: Yebo Li, Wooster, OH (US); Yuguang Zhou, Beijing (CN)

(21) Appl. No.: 13/040,256

(22) Filed: Mar. 3, 2011

Related U.S. Application Data
(63) Continuation-in-part of application No. 12/632,433, filed on Dec. 7, 2009, now Pat. No. 8,022,257.
(60) Provisional application No. 61/239,581, filed on Sep. 3, 2009.

(51) Int. Cl. C08G 18/00; C07C 29/00

(52) U.S. Cl. 528/85; 568/852

(57) ABSTRACT

Methods for producing a polyols and polyurethanes are described. The polyols described herein can be produced directly from crude glycerin or through liquefaction of lignocellulosic biomass using a solvent comprising crude glycerin. The polyols produced in accordance with certain aspects may be derived from a significant proportion of renewable resources.

This application is a continuation-in-part of application Ser. No. 12/632,433, filed Dec. 7, 2009, which claimed benefit of U.S. application Ser. No. 61/239,581, filed Sep. 3, 2009, the contents of both of which are hereby incorporated by reference.

### BACKGROUND OF THE INVENTION

The present application relates to methods for producing polyols and polyurethanes, and more particularly, to producing polyols from lignocellulosic biomass using a solvent containing crude glycerin, and to producing polyols from crude glycerin without lignocellulosic biomass. The resulting polyols can be used as starting materials for various products and in particular the polyols can be used to produce polyurethanes.

The industrial manufacturing of polyurethane foam and elastomer products rely significantly on fossil fuels and its derivatives as major reactants for the production of polyols. Certain aspects of the present application are directed to methods of manufacturing polyurethanes using materials that are in abundant supply and obtained from renewable resources. By utilizing renewable resources instead of relying on limited supplies of fossil fuels and derivatives, some aspects of the present invention provide methods for manufacturing polyurethane products through an environmentally friendly process that is also characterized by reduced production costs because the raw materials are readily available byproducts or waste products.

The major global consumption of polyurethane products is in the form of foams which come in two types, flexible and rigid, being roughly equal in market size. In 2007, the global consumption of polyurethane (PU) raw materials was above 12 million metric tons, the average annual growth rate is about 5%. Polyurethane products are used in many different ways as summarized in Table 1 below.

TABLE 1
Polyurethane products consumption (US Data 2004)

| | Amount of | |
|---|---|---|
| | polyurethane used | Percentage of |
| Application | (millions of pounds) | total |
| Building & | 1,459 | 26.80% |
| Construction | | |
| Transportation | 1,298 | 23.80% |
| Furniture & | 1,127 | 20.70% |
| Bedding | | |
| Appliances | 278 | 5.10% |
| Packaging | 251 | 4.60% |
| Textiles, Fibers | 181 | 3.30% |
| & Apparel | | |
| Machinery & | 178 | 3.30% |
| Foundry | | |
| Electronics | 75 | 1.40% |
| Footwear | 39 | 0.70% |
| Other uses | 558 | 10.20% |
| Total | 5,444 | 100.00% |

The general polyurethane polymer-forming reaction between an isocyanate as the A-side component and an alcohol or polyol as the B-side component is as follows:

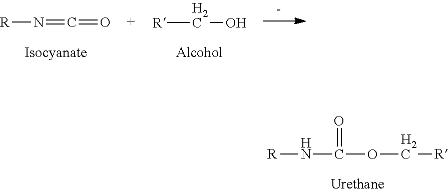

### Typical Reaction of Urethane Formation

One source of crude glycerin is as a byproduct and waste from the transesterification process of biodiesel production. Biodiesel, produced according to ASTM D 6751, is known as a mono-alkyl methyl ester (fatty acid methyl ester, FAME or MEFA, methyl ester of fatty acid) or methyl ester for short. It can be made from multiple sources (waste vegetable oil, soybean oil, canola oil, sunflower oil, corn oil, flaxseed oil, cottonseed oil, peanut oil, lard, grease, poultry fat, cooking oil, algae etc.). Crude glycerin derived from biodiesel production has very low value because of its impurities. Generally, crude glycerin appears as a brown liquid since it contains glycerin, methanol, sodium hydroxide, moisture, and some fat residues.

### SUMMARY OF THE INVENTION

The present application is directed to methods for producing polyols from crude glycerin, especially with most impurities remaining in the crude glycerin. In accordance with one aspect, polyols can be produced from crude glycerin itself. In accordance with another aspect, polyols can be produced through the liquefaction of lignocellulosic biomass using a solvent comprising crude glycerin. In accordance with one aspect, lignocellulosic biomass is combined with crude glycerin to form an admixture and the admixture is heated to liquefy the biomass and produce a polyol.

In accordance with other embodiments, the polyol produced from the liquefied lignocellulosic biomass or the polyol produced from crude glycerin itself can be used as a reactant in a method for producing polyurethane.

The present application is also directed to the polyols and polyurethanes produced in accordance with the described methods. These and other objects, advantages and features of the invention will be apparent from the following description of a preferred embodiment, considered along with the accompanying drawings.

### DESCRIPTION OF THE DRAWINGS

FIG. 1 is a schematic diagram illustrating one embodiment of the present invention related to the production of biopolyols and polyurethanes from the crude glycerin byproduct of the biodiesel production process.

FIG. 2 is another schematic diagram indicating a variation of the invention.

FIG. 3 is a graph showing the effect of adding unrefined crude glycerin to pure glycerin on the polyol hydroxyl value. All data points included about 10 wt. % biomass in the formulation. Additional points are included using 100% of the unrefined crude glycerin as feedstock for the polyol, as well as blends of pure glycerin with soap and soap plus biodiesel.

FIG. 4 shows the effect of the total glycerin content on the hydroxyl number for polyols made without any biomass.

FIG. 5 shows the effect of the total glycerin content on the viscosity of the polyols made without any biomass.

### DESCRIPTION OF PREFERRED EMBODIMENTS

The present application is directed to methods for producing polyols utilizing crude glycerin. Polyols can be produced directly from the crude glycerin or through liquefaction of lignocellulosic biomass using a solvent comprising crude glycerin. In accordance with certain aspects of the present invention, polyols are produced in accordance with methods that utilize non-petroleum and also preferably non-food feedstock, some of which are considered waste byproducts. Polyols produced in accordance with the methods described herein can be considered highly functional polyols that can be used as a reactant in various reactions, including as a reactant in a method for producing polyurethane. In accordance with some aspects, the resulting polyols can be used directly for producing polymers.

Isolation or purification of the polyols is not required. Biomass, a renewable energy source, is biological material derived from living, or recently living organisms. It also includes lignocellulosic plant materials such as corn stover, wheat bran, rice stalk, soybean straw, herbals and wood sawdust and similar materials.

In accordance with one embodiment, lignocellulosic biomass is used as one part of the raw material for liquefaction as described herein. Generally, lignocellulosic biomass refers to agricultural wastes (corn stover, wheat straw, rice stalk, soybean straw, etc.), wood wastes (dead trees, wood chips, sawdust, etc.), and other types of plants, industrial paper pulp, herbals and so on.

The lignocellulosic biomass material typically is reduced in size to facilitate liquefaction. In accordance with certain aspects, the material can be chopped into pieces. It can also be milled to provide the appropriate size particles. The liquefaction process in accordance with certain embodiments utilizes biomass particles below about 50 mm, more particularly below about 20 mm.

In accordance with one aspect, crude glycerin obtained as a byproduct of biodiesel production is used as solvent in the liquefaction process described herein. Crude glycerin typically contains glycerin, methanol, inorganic salts such as sodium hydroxide or potassium hydroxide, water, oils or fat, soap, residual biodiesel (MEFA/FAME) and so on. As used herein, “crude glycerin” refers to a glycerin component that is obtained as a by-product of a reaction and, in particular, as a by-product of a reaction for producing biodiesel fuel (BDF). Crude glycerin as produced during the production of BDF typically has a glycerin content of about 38-88% and which can include partial refining to remove or reduce impurities such as methanol, water, salts and soaps. Partial refinement can increase the glycerin content up to about 90% glycerin, more particularly up to about 95% glycerin and in certain cases up to about 97% glycerin, approaching the purity associated with technical grade glycerin or “pure glycerin”. As used herein, crude glycerin includes crude glycerin as produced and crude glycerin that has been partially refined to a purity near that for technical grade glycerin. The term “unrefined crude glycerin” refers to crude glycerin as produced during a reaction such as BDF production in which the byproduct as not been refined other than being treated to remove methanol (which occurs by boiling off the methanol on initial heating) and large impurities such as the fat and soap layer removable by skimming. Crude glycerin from a particular biodiesel production process utilizing NaOH as a catalyst was analyzed to determine levels of other elements/metals. Na was found to be of highest concentration (18,300 μg/g average). Other metals of relatively higher concentration include K (79 μg/g average, Ca (18 μg/g average, Mg (5.2 μg/g average). Other metals/elements were present in trace amounts. The glycerin by-product from the NaOH catalyzed biodiesel process may contain at least 5000 μg/g, more particularly at least 10,000 μg/g, and in certain cases at least 15,000 μg/g sodium.

Analysis of crude glycerin feedstock from several biodiesel production sources showed, glycerin content of about 38.5% to 39% by weight, total fatty acid/soap content of about 36% to 49% by weight, about 11% methanol by weight and about 3% to 6% water by weight of total crude glycerin. MEFA (biodiesel) was also present due to imperfect separation of MEFA from crude glycerin in the biodiesel process. In the reaction step, the methanol and moisture will be removed. The resulting feedstock will have about 48% glycerin, 52% total fatty acid which includes soaps, MEFA, and free fatty acids.

As used herein, the terms “glycerin,” “glycerine,” or “glycerol” are used interchangeably and refer to the compound 1,2,3, propanetriol. The terms “biopolyol” and “bio-based polyol” refer to polyols synthesized from bio-based materials such as agricultural biomass, vegetable oils or animal fats and byproducts of the biodiesel process. Polyols produced in accordance with certain embodiments may be derived from at least 70%, more particularly at least 80%, and in some cases at least 90% from renewable resources. In still other cases the polyols may be derived from substantially all renewable resources. The term “substantially” allows for small amounts (<1-2%) of non-renewable materials. The term “renewable resources” as used herein is intended to mean non-petroleum sources.

Although the economic benefits of certain aspects of the present invention are related to the use of low-cost crude glycerin, some portion of the solvent mixture can include refined (>97% purity) glycerin or other solvents. In accordance with certain embodiments, the amount of refined glycerin or other solvent would be no more than 50%, more particularly no more than 20%, no more than 10% or no more than 5%, by weight of the total solvent by weight. In accordance with other embodiments, the solvent comprises substantially 100% crude glycerin. The term “substantially” allows for trace amounts (less than 2%) of other solvents or components besides the crude glycerin. In accordance with still other embodiments, the solvent consists essentially of crude glycerin product as obtained from the generating source.

The reaction for producing BDF may include a transesterification reaction or alcoholysis reaction that occurs in a basic reaction mixture (e.g., having a pH greater than about 11) comprising triglycerides (e.g., which are present in animal or vegetable fats or oils) and alcohol (e.g., methanol or ethanol). The reaction mixture may produce fatty acid alkyl esters (e.g., fatty acid methyl esters) and glycerin. As indicated above, the fat and soap layer from the crude glycerin may be skimmed to get rid of floating impurities. The main liquefaction process can be performed in either pressurized or atmospheric conditions. Biomass and solvent, primarily crude glycerin, are well mixed in a biomass to solvent mass ratio range sufficient to effectively liquefy the biomass. Typically, the ratio of biomass to solvent mass is about 0.05-20%, more specifically about 0.5-15%, and in certain cases about 5-10%. The ratio is expressed as a percentage equivalent to the mass of biomass divided by the mass of solvent.

In accordance with certain embodiments, polyols can also be produced from crude glycerin in the absence of separately provided biomass. Polyols produced directly from crude glycerin can also be used to produce polyurethanes in accordance with certain aspects of the present invention. The process for producing polyols from crude glycerin without biomass is essentially the same as the process described herein relating to liquefaction of biomass in a solvent containing crude glycerin.

The liquefaction process typically is conducted at an elevated temperature for an amount of time effective to produce the polyol from the biomass and solvent. Typical reaction conditions include temperatures of 130° C. to 220° C. for about 10 minutes to about 10 hours. In accordance with certain embodiments, the reaction may be conducted in a temperature range of about 180° C. to 215° C., more particularly about 185 to 210° C. and in accordance with certain embodiments about 190-200° C.

A catalyst (acid, base, or salt) may also be used and can be processed with or without continuous blending. Examples of catalysts that may be used include, without limitation, H₂SO₄, CH₃COOK, CH₃COOH, HCl, H₃PO₄, HCOOH, ZnCl₂, NaOH, KOH, Na₂CO₃, K₂CO₃, Rb₂CO₃, Cs₂CO₃, Ni, Cu(OH)₂, NaHCO₃, NaAc, MgAc, NH₄Cl, ZnCl₂, AlCl₃, (NH₄)₂SO₄, FeSO₄, Fe(NH(SO₄)₂, ZnSO₄, CaSO₄, MgSO₄, NaH₂PO₄, Na₂HPO₄, etc. Typically, catalysts may be used at a concentration of about 0.5-10%, more particularly about 1-5%. When concentrated sulfuric acid is used, the sulfuric acid concentration may be about 2-6%.

The liquefaction process is continued for a time sufficient to sufficiently convert the lignocellulosic biomass to a polyol composition. In certain conditions, the liquefaction lasted for less than 90 minutes. Typically, a black-colored, semi-fluid product is obtained after liquefaction.

In accordance with certain aspects of the present invention, the liquefaction process results in a biomass conversion of at least 70%, more particularly at least 80%, still more particularly at least 90% and in some cases at least 95%. The percent conversion quantifies the amount of insoluble residue remaining after the liquefaction process. The percent conversion can be measured by dissolving a weighed sample of the polyol product in a 4:1 v/v dioxane-water solution and letting the dissolved sample stand for 24 hours without stirring. After standing, the solvent is filtered out and the sample is washed with solvent until clear. The insoluble material on the filter paper is dried overnight in an oven at 105° C. and then weighed. The % conversion can be calculated using the following equation:

% conversion=[1−(Wr/Ws)]×100

where Ws is the weight of the initial sample and Wr is the weight of the insoluble residue, thus expressing the conversion as a yield percentage essentially comparing the weight of polyol produced to the weight of the initial admixture including solvent and biomass.

In accordance with a particular embodiment, the liquefaction setup includes a three-necked round-bottom flask connected to a reflux condenser. A 500-mL three-necked round-bottom flask fitted with a reflux condenser, a heater, thermometer, and mechanical stirrer is set up under a fume hood. The pre-weighed lignocellulosic biomass, crude glycerin, and catalyst are added into the flask and heated to an elevated temperature. Alternatively, the solvent and catalyst can be added to the flask, heated to the reaction temperature and then the biomass is added. The reflux condenser is provided with a gas port connected to a rubber tubing into a bottle where the condensing methanol-rich steam is collected. Methanol can be recovered through the reflux condenser at temperatures up to 130° C. The recovery ratio of methanol is dependent on the methanol content of the crude glycerin, which typically is in the range of about 10˜20% but can vary depending on the biodiesel process and the starting materials.

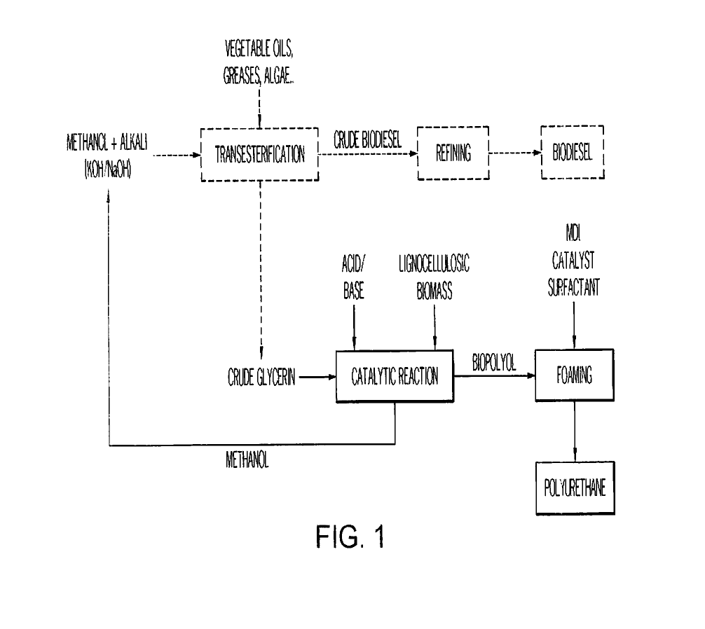

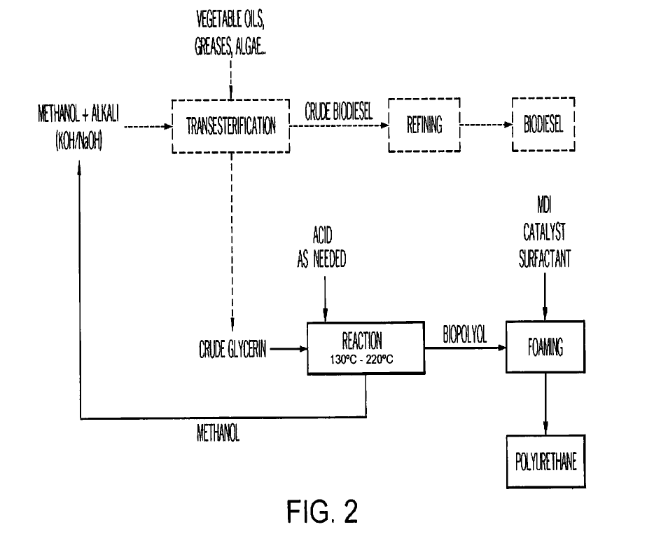

As noted above, in another embodiment of the invention a polyol is produced without biomass. The procedure is essentially the same as described above, but without addition of lignocellulosic biomass and without actual liquefaction of any component. FIG. 2 schematically shows the process, indicating, like FIG. 1, the transesterification of oils to produce biodiesel fuel, the process from which the byproduct crude glycerin is obtained. The crude glycerin, preferably unrefined crude glycerin as defined above, or a glycerin that includes similar components, is heated to a temperature of at least 130° C., more preferably at least 150° C. and optimally at least 180° C. or at least 185° C. An upper limit of temperature has not been firmly established, but the temperature should generally be below 300° C. and more preferably no higher than about 260° C. Temperatures of 220° C. to 240° C. are effective, especially where biomass is liquified. If no biomass is included, temperatures of 185° C. to 200° C., or about 190° C., are sufficient.

For laboratory purposes, the heating of the crude glycerin can be as described above, with a three-necked round-bottomed flask connected to a reflux condenser, or the process can be conducted on a much larger, commercial scale. The crude glycerin preferably is heated in air, and as the temperature of the liquid rises, well below the maximum temperature, methanol boils off and can be collected in the reflux condenser. The methanol can be reused as indicated in FIGS. 1 and 2.

The resulting biopolyol is useful in producing polyurethanes, including polyurethane foams, by a reaction with isocyanates as described below.

Examples are presented below. These examples reflect tests conducted using unrefined crude glycerin, as is preferred, blends of pure glycerin and unrefined crude glycerin, such blends with methyl esters of fatty acids (biodiesel) added, blends with soap/fatty acids, and also using pure glycerin without “contaminants”. In some of the examples biomass was included, liquified in the process, and in other examples biomass was omitted. The tests show that in all cases, the fatty acid/soap content is essential to produce a quality, useful polyol that can be reacted with isocyanate to produce a polyurethane. As noted below, the examples indicate a range of fatty acid/soap content that should be present for optimum results. Such optimum results primarily relate to hydroxyl number of the polyol, but also other characteristics. The examples show that pure glycerin, without fatty acid/soap is not effective in producing a useful polyol. In all tests the reaction temperature was in the range of essentially 220° C. to 240° C., although temperatures in a wider range can be effective.

### Polyols from Liquefaction of Biomass

Tests were performed to produce polyols and test polyols as made from (a) pure glycerin, (b) pure glycerin contaminated with the soap/fatty acid and/or methyl ester of fatty acid (MEFA, i.e. biodiesel) taken from unrefined crude glycerin (URCG), (c) unrefined crude glycerin, and (d) blends of pure glycerin and unrefined crude glycerin. In these tests involving liquefaction of biomass, a three-necked flask was used as described above, and the admixture was brought to a temperature in the range of about 220° C. to 240° C., at atmospheric pressure. Methanol contained in the unrefined crude glycerin was boiled off at temperatures far below the reaction temperature. In some tests acid was added and in some tests none was added, as noted in the table below in the various examples.

Analysis of two samples of unrefined crude glycerin from two different sources by a third party laboratory found that the unrefined crude glycerin contained about 39% by weight glycerin, 11.2% to 11.4% by weight methanol, 3% to 6% moisture and 36% to 49% by weight total fatty acid. The fatty acid percentage includes fatty acid and soaps (which are salts of fatty acids). A surface layer of soap/fatty acid had been skimmed off the unrefined crude glycerin before testing. Once the methanol and water had boiled off, which occurs early in the heating process, the remaining unrefined crude glycerin had somewhat higher proportions of crude glycerin and total fatty acid, about 48% and 52%, respectively, of the remaining unrefined crude glycerin without methanol and water.

Polyurethane foams were produced, where possible, from polyols made from:

a) pure glycerin,

b) pure glycerin contaminated with about 60% of the soap/fatty acids and/or MEFA skimmed off of the URCG,

c) URCG, and

d) blends of pure glycerin and URCG.

These polyurethane foams were made by blending in a predetermined amount of isocyanate such that the isocyanate level resulted in an Index of 100. Impurities (soap/fatty acid and MEFA) were added into pure glycerin (samples b) to simulate the composition of URCG as determined by the third party laboratory in Tennessee. Additionally, experiments were performed mixing pure glycerin with unrefined crude glycerin (samples d) as the feedstock for making the polyol. These experiments allowed the amount of glycerin to be varied. For polyurethane foams, the term index is used to determine the amount of isocyanate to add to a polyol. Index is defined as the actual amount of isocyanate used divided by the theoretical amount needed×100. At an index of 100, the isocyanate level is at the theoretical optimum level for reaction. The foaming research work was done at an isocyanate index of 100. Using this constant index level of isocyanate in the polyol, the foaming characteristics of various polyols made from pure glycerin, “contaminated glycerin” or glycerin with impurities, unrefined crude glycerin, and blends of pure and unrefined crude glycerin were compared.

Unrefined crude glycerin successfully liquefied biomass in the presence of 3 wt. % sulfuric acid at 220° C. for 90 minutes and produced a flowable liquid polyol of good quality.

When the polyols produced with URCG were blended with catalysts and surfactant with water as blowing agent or blend to react with polymeric MDI at isocyanate index of 100, a continuous and a good quality foam product was produced.

When pure glycerin was used in place of the URCG to liquefy biomass in the presence of 3 wt. % sulfuric acid and heated at the same temperature and time as noted above, the result was not a free flowing liquid, but a clumped, non-flowing product. This could not be used to form a foamed polyurethane material. The comparison of these two samples clearly shows that polyol made from crude glycerin is very different from that made with pure glycerin.

Additional testing showed that the acid had a negative effect when used with pure glycerin. When the experiment as described immediately above was repeated without acid and heated at the same temperature and time as noted above for unrefined crude glycerin, the resultant polyol was used to make foam, but the foam had very poor structure, with discontinuity and voids, and was an unacceptable product.

The following table supports these conclusions.

| | | | | | |
|---|---|---|---|---|---|
| | | | | Hydroxyl | Foam |
| Example | | Acid | Biomass | Value | Density |
| No. | Sample | (wt. %) | (wt. %) | (mg KOH/g) | (g/cm3) |

Pure Glycerin with Unrefined Crude Glycerin Blends

| | | | | | |
|---|---|---|---|---|---|
| 1 | Pure Glycerin | 0 | 10 | 1622 | 0.144 |
| | (100 wt. % Total Glycerin) | | | | |
| 2 | 81 wt. Pure Glycerin, 19 wt. % Unrefined Crude | 0 | 10 | 1370 | 0.081 |
| | Glycerin (88 wt. % Total Glycerin) | | | | |
| 3 | 70 wt. Pure Glycerin, 30 wt. % Unrefined Crude | 0 | 9.9 | 1424 | 0.096 |
| | Glycerin (82 wt. % Total Glycerin) | | | | |
| 4 | 50 wt. % Pure Glycerin, 50 wt. % Unrefined | 0 | 10 | 1107 | 0.058 |
| | Crude Glycerin (69 wt. % Total Glycerin) | | | | |
| 5 | 31 wt. % Pure Glycerin, 69 wt. % Unrefined | 0 | 10 | 977 | 0.081 |
| | Crude Glycerin (57 wt. % Total Glycerin) | | | | |

Pure Glycerin with Soap and Fatty Acids

| | | | | | |
|---|---|---|---|---|---|
| 6 | 60 wt. % Pure Glycerin, 40 wt. % Soap | 1.0 | 10 | 918 | 0.050 |
| | skimmed off of URCG (60 wt. % Total Glycerin) | | | | |
| 7 | 40 wt. % Pure Glycerin, 60 wt. % Soap | 0 | 10 | 748 | 0.053 |
| | skimmed off of URCG [sample approximates | | | | |
| | URCG by mixture] (40 wt. % Total Glycerin) | | | | |
| 8 | 39 wt. % Pure Glycerin, 41 wt. % Soap and 20 | 0 | 10 | 667 | 0.050 |
| | wt. % Methyl Ester of Fatty Acids (MEFA) | | | | |
| | (Soap and MEFA from URCG) (39 wt. % Total | | | | |
| | Glycerin) | | | | |

Unrefined Crude Glycerin

| | | | | | |
|---|---|---|---|---|---|
| 9 | Unrefined Crude Glycerin (39 wt. % Total | 3.1 | 10 | 573 | 0.034 |
| | Glycerin) | | | | |
| 10 | Unrefined Crude Glycerin (39 wt. % Total | 0 | 10 | 609 | 0.091 |
| | Glycerin) | | | | |

Note that a mixture of pure glycerin with biomass and with acid did not make a polyol that could be reacted with isocyanates to form a foamed material.

Acid and biomass percentages are calculated as grams acid or biomass divided by grams of solvent (pure glycerin, URCG, soap, MEFA) times 100%. Examples 1-4 are comparative examples while Examples 5-12 are examples of the invention.

In examples 8 and 10 above, 20% and 40% by weight MEFA (biodiesel), respectively, were included with the soap and pure glycerin, because varying amounts of MEFA are present in the crude glycerin feedstock due to imperfect separation the biodiesel from the byproduct. Example 9 consists of pure glycerin and only MEFA, no soap. This blend made an acceptable polyol and an acceptable foam.

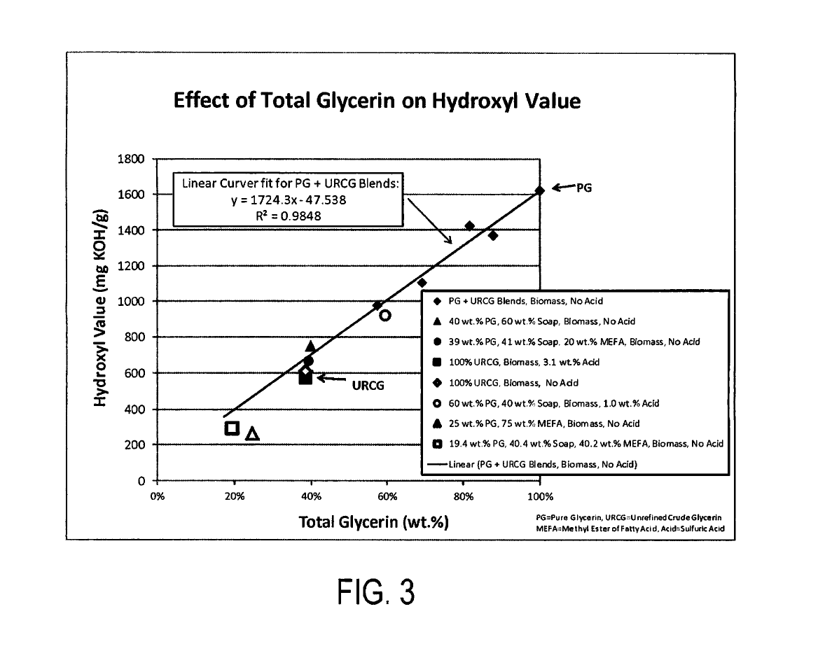

The graph of FIG. 3 shows the effect of adding URCG to pure glycerin on the polyol hydroxyl value. All points in FIG. 3 contained about 10 wt. % biomass. The points of the linear-fit line are in the table above. Additionally, there are data points included for 100% unrefined crude glycerin as well as mixtures of pure glycerin with soap, and pure glycerin with soap and MEFA, both selected to simulate an unrefined crude glycerin mixture. These three mixtures, all at about 40% glycerin, produced a polyol with similar hydroxyl values, all much lower (at least 50% less) than the hydroxyl value from the polyol from the pure glycerin. An additional point at 60 wt. % total glycerin with acid is also shown on the graph.

In the production of urethane foams, polyols are used that have a hydroxyl value (mg KOH/g) less than about 1000, and usually less than 800. From the data and FIG. 3, it is clear that when fatty acids and soaps are present in the reactor vessel with glycerin, suitable polyols are made. The soap and fatty acids are incorporated into the glycerin molecule, creating a long chain molecule. As more soap and/or fatty acid are incorporated into the glycerin molecule, the molecular weight of the polyol product will increase, most likely without increasing the functionality or number of reactive sites per molecule. As the molecule grows, and thus the molecular weight grows, via reactions with the soap/fatty acid and/or MEFA, the number of reacting sites per gram of polymer decreases. As the number of reacting sites per gram of polyol decreases, the hydroxyl value will decrease. Additionally, as the polyol molecule increases in size, the reacting sites, or hydroxyls, will be separated from each other spatially. As this separation increases, the polymer chain structure between hydroxyl sites becomes more flexible, and the ability for the bulky isocyanate molecule, often diphenylmethane diisocyanate (MDI) or toluene diisocyanate (TDI), to react with the hydroxyl site improves.

In an unrefined crude glycerin, or a pure glycerin admixture contaminated with fatty acids/soaps and/or MEFA, with biomass in the admixture at about 5% to 20%, a preferred range of glycerin composition or content is about 19% to 60% by weight of the total, the balance essentially being fatty acid/soap and optionally MEFA, the total being exclusive of any methanol present or water. A more preferred range is about 25% to 55% glycerin, essentially the balance being fatty acids/soaps and optionally MEFA. In another preferred embodiment the glycerin content is not greater than about 50% of the total URCG by weight excluding any methanol. Stated another way, a preferred embodiment has the crude glycerin or synthesized crude glycerin with at least about 45% by weight fatty acids/soaps and/or MEFA disregarding any methanol present. More preferably this percentage is at least about 50%.

Note that acid content has an influence on the reaction to produce a polyol. With unrefined crude glycerin, Examples 11 and 12, the addition of acid to the URCG caused a lower hydroxyl number and a better foam density, about 0.03 g/cm³ versus 0.09 g/cm³. When pure glycerin was used, either alone or with additives, results tended to be better without addition of acid. Feedstock unrefined crude glycerin had a pH typically in the range of about 8 to 9.5, and this pH was found advantageously adjusted to about 6 to 8, more preferably 6.5 to 7.5, by addition of an appropriate amount of acid when necessary. Control of acidity by addition of acid as noted above was also found to reduce viscosity of the resulting polyol. A generally desirable range of viscosity of the polyol was about 100-50,000 cP at 25° C., more preferably about 100-10,000 cP or 300-10,000 cP. When biomass is converted in the reaction, acid addition tends to lower viscosity of the polyol.

### Polyols Produced from Crude Glycerin without Biomass

Further tests were conducted under similar parameters as described above, but without biomass, producing polyols from pure glycerin, blends of pure glycerin and unrefined crude glycerin (URCG), blends of pure glycerin with fatty acid/soap and/or MEFA, and crude glycerin alone or combined with soap.

In these tests, reported in the examples below, reaction temperature was in the range of about 190° C. to 210° C. Without biomass present, the reaction temperature range can be somewhat lower than the case where biomass is liquefied. The following table lists the effect of reaction temperature when unrefined crude glycerin with 5 wt. % acid is heated for various time periods. It is clear that as the reaction temperature is increased, the reaction proceeds, lowering the acid number as well as lowering the hydroxyl number.

### Effect of Temperature—No Biomass

| | | | | | | |
|---|---|---|---|---|---|---|
| | | | | Acid | | |
| | Temper- | | Sulfuric | number | Hydroxyl | |
| Ex- | ature | Reaction | acid | (mg | number | Viscosity |
| ample | (?) | time (min) | (wt. %) | KOH/g) | (mg KOH/g) | (cp) |

Feedstock: Unrefined Crude Glycerin and Acid

| | | | | | |
|---|---|---|---|---|---|
| 13 | 150 | 90 | 30 | 813 | 564 |
| 14 | 170 | 90 | 30 | 774 | 540 |
| 15 | 190 | 90 | 15 | 635 | 468 |
| 16 | 210 | 90 | 13 | 604 | 960 |

When pure glycerin is blended with URCG without biomass and without acid, as shown in the following table, as listed in examples 17 to 21, the hydroxyl value decreased to an acceptable level, but the polyol viscosity increased dramatically when the URCG was 75% or more in the blend by weight. When acid was used in the reaction, as listed in examples 22 to 24, the hydroxyl value increased, but the viscosity was significantly lower as compared to the examples 17 to 21. Also, the foam density was lower when acid is used with URCG.

### Pure Glycerin and Crude Glycerin Mixture—No Biomass

| | | | | | Hydroxyl | Acid | | |
|---|---|---|---|---|---|---|---|---|
| | Total | Pure | Crude | 98% | Number | Number | | Foam |
| | Glycerin | Glycerin | Glycerin | H₂SO₄ | (mg | (mg | Viscosity | Density |
| Example | (wt. %) | (wt. %) | (wt. %) | (wt. %) | KOH/g) | KOH/g) | (cP) | g/cm3 |
| 17 | 100% | 100% | 0% | 0% | 1509 | 0.9 | 2965 | 0.090 |
| 18 | 84% | 75% | 25% | 0% | 1463 | 1.1 | 2351 | 0.082 |
| 19 | 69% | 50% | 50% | 0% | 1104 | 0.7 | 2697 | 0.063 |
| 20 | 54% | 25% | 75% | 0% | 804 | 1.5 | >100,000 | 0.077 |
| 21 | 39% | 0% | 100% | 0% | 495 | 7.1 | >100,000 | 0.076 |
| 22 | 69% | 50% | 50% | 3% | 1306 | 13.7 | 294 | 0.064 |
| 23 | 54% | 25% | 75% | 3% | 972 | 7.1 | 684 | 0.052 |
| 24 | 39% | 0% | 100% | 3% | 615 | 29.0 | 744 | 0.046 |

When pure glycerin was mixed with soap/fatty acid without acid, the hydroxyl values decreased as the soap was increased. When about 60% soap was used in pure glycerin by weight, the resultant polyol had acceptable viscosity and acid numbers and made an acceptable foam as shown in the following table.

### Pure Glycerin And Soap Mixture—No Biomass

| | | | | | Hydroxyl | Acid | | |
|---|---|---|---|---|---|---|---|---|
| | Total | Pure | Soap/ | 98% | Number | Number | | Foam |
| | Glycerin | Glycerin | Fatty Acid | H₂SO₄ | (mg | (mg | Viscosity | Density |
| Example | (wt. %) | (wt. %) | (wt. %) | (wt. %) | KOH/g) | KOH/g) | (cP) | (g/cm3) |
| 25 | 80% | 80% | 20% | 0% | 1389 | 0.2 | 3131 | 0.062 |
| 26 | 60% | 60% | 40% | 0% | 976 | 0.9 | 3299 | 0.068 |
| 27 | 40% | 40% | 60% | 0% | 675 | 1.1 | 5093 | 0.051 |
| 28 | 20% | 20% | 80% | 0% | 719 | 0.0 | 4094 | 0.060 |
| 29 | 0% | 0% | 100% | 0% | 568 | 0.6 | 518.3 | 0.105 |
| 30 | 67% | 67% | 33% | 1.50% | 1329 | 5.8 | 3527 | 0.070 |

Examples 31 to 36 below list the results of blending MEFA into pure glycerin without acid. The MEFA acts as a diluting agent to the significantly reduce the polyol viscosity when the MEFA was above 15 wt. %. Even below this 15 wt. % concentration, the viscosity was very acceptable.

### Pure Glycerin and MEFA

| | | | Methyl | | | | | |
|---|---|---|---|---|---|---|---|---|
| | | | Ester of | | Hydroxyl | Acid | | |
| | Total | Pure | Fatty | 98% | Number | Number | | Foam |
| | Glycerin | Glycerin | Acids | H₂SO₄ | (mg | (mg | Viscosity | Density |
| Example | (wt. %) | (wt. %) | (wt. %) | (wt. %) | KOH/g) | KOH/g) | (cP) | (g/cm3) |
| 31 | 85% | 85% | 15% | 0% | 1417 | 0.41 | 989.8 | 0.0909 |
| 32 | 60% | 60% | 40% | 0% | 1386 | 1.02 | | 0.0879 |
| 33 | 50% | 50% | 50% | 0% | 1217 | 0.01 | 15.4 | * |
| 34 | 40% | 40% | 60% | 0% | 1163 | 1.25 | 12 | * |
| 35 | 30% | 30% | 70% | 0% | 1035 | 1.19 | 23.5 | * |
| 36 | 0% | 0% | 100% | 0% | 11 | 1.96 | 12.1 | 0 |

When soap or MEFA was added to crude glycerin, as shown below in the following table, examples 37 and 38, the hydroxyl values were within this invention and had very low viscosity values.

### Crude Glycerin, Soap and Methyl Ester of Fatty Acids Mixture

| | | | | Methyl | | Hydroxyl | Acid | |
|---|---|---|---|---|---|---|---|---|
| | Total | Crude | | Ester of | | Number | Number | |
| | Glycerin | Glycerin | Soap | Fatty Acids | 98% | (mg | (mg | Viscosity |
| Example | (wt. %) | Wt. (%) | wt. (%) | (wt. %) | H₂SO₄ (%) | KOH/g) | KOH/g) | (cP) |
| 37 | 24% | 50% | 50% | 0% | 0 | 623 | 10.2 | 17 |
| 38 | 24% | 50% | 0% | 50% | 0 | 658 | 17.2 | 19 |

When pure glycerin was mixed with various levels of soap and MEFA, the polyol from the mixed material was able to be used to make a foam, and had acceptable viscosity values, acid number values. The viscosity lowering effect of the acid is shown below when example 40 is compared to example 39.

### Pure Glycerin, Soap and Ester of Fatty Acids Mixture

| | | | | Methyl | | Hydroxyl | Acid | | |
|---|---|---|---|---|---|---|---|---|---|
| | Total | Pure | | Ester of | 98% | Number | Number | | Foam |
| | Glycerin | Glycerin | Soap | Fatty Acids | H₂SO₄ | (mg | (mg | Viscosity | Density |
| Example | (wt. %) | (wt. %) | wt. (%) | (wt. %) | (%) | KOH/g) | KOH/g) | (cP) | (g/cm3) |
| 39 | 50% | 50% | 30% | 20% | 0.5% | 1132.9 | 2.83 | 366 | 0.0714 |
| 40 | 41% | 40% | 40% | 20% | 0% | 979.5 | 1.38 | 1272 | 0.0598 |

The above examples show that the addition of acid can be an important factor. The example with pure glycerin alone, no acid added, produces a polyol with acid number 0.9 KOH/g. All of the examples that included pure glycerin in any amount were without addition of acid, yet nearly all produced polyols with positive acid numbers. In the case of crude glycerin, acid is important in reducing viscosity, as shown in Examples 13 through 16, 37 and 38, as well as Examples 20, 21, 23 and 24. The feedstock unrefined crude glycerin is from a variety of biodiesel production processes, that use various oils such as soybean oil, used cooking oil, vegetable oils and other oils. The feedstock usually has a pH in the range of about 8 to 9.5. For production of good polyols it is usually desirable to bring the pH down, but addition of acid, to a range of about 6.0 to 8.0. More particularly, a range of pH 6.5 to 7.5 is preferred. The examples where useful polyols were produced show addition of 3% acid (by weight as compared to total solvent which includes glycerin, soaps and MEFA if present). The testing tends to show that if any substantial portion of pure glycerin is included (e.g. about 40% or more), and crude glycerin is not included, very little if any acid should be added, e.g. 1.5% by weight of total solvent or less.

On the other hand, where all crude glycerin (URCG) was used, as in Examples 37 and 38 acid was needed to reduce viscosity. Examples 20 and 21, with URCG content of 75% by weight and 100% by weight of solvent, showed viscosities of over 100,000 cP. These tests were without acid. Foams were produced from these highly viscose polyols, but the foams were hollow and not consistent, not acceptable foams.

The examples show a reduction of hydroxyl number with increased amount of crude glycerin (URCG) and less pure glycerin in the total solvent. This trend is shown in Examples 17, 18, 22, 19, 23, 20, 24 and 21. At the same time, the examples where acid was added show a slight to moderate increase in hydroxyl number with the acid. Hydroxyl numbers of over 1000 are generally not acceptable, although they can be used for some purposes. Hydroxyl number for polyols producing rigid foams can be up to 800 or 900 or even about 975, but are more preferably 800 or lower.

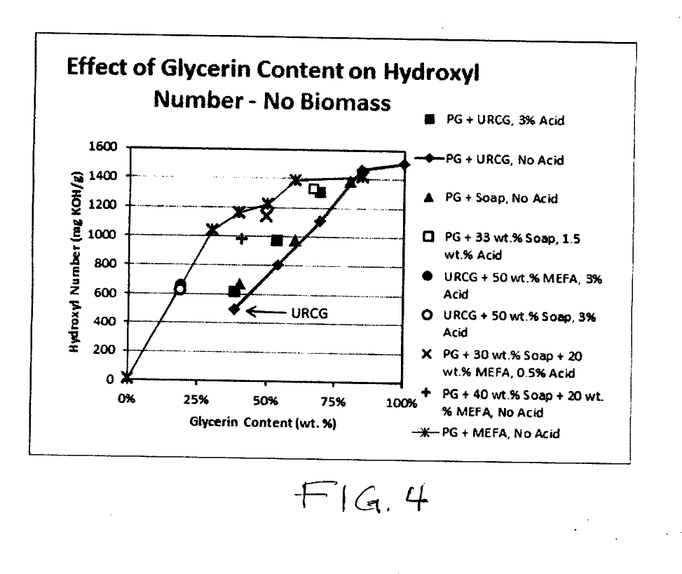

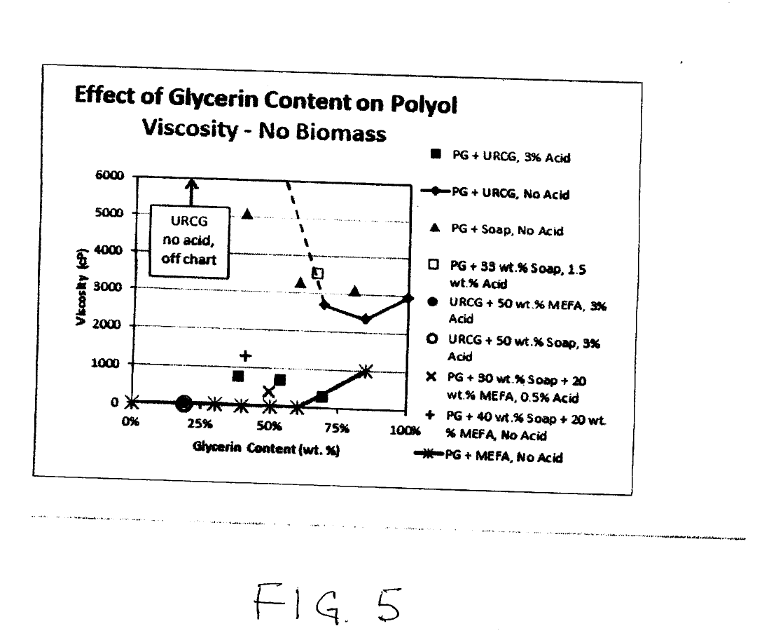

FIG. 4 clearly shows the effect of using URCG in place of pure glycerin when making polyols by this process. As the amount of total glycerin content is reduced, the hydroxyl number reduces from a range that is too high to make acceptable foam into a range that is needed to make polyurethane products. FIG. 5 shows the viscosity effect discussed above. For compositions containing no biomass, with acid, the viscosity decreased as the glycerin is decreased. For examples without acid, the figure clearly shows how the viscosity increased as the glycerin was decreased.

Viscosity in a good, useful polyol should be under about 50,000, and preferably lower. Viscosity greatly decreases with temperature, so that the relative high-viscosity polyols can be used to produce useful polyurethanes when the polyol is heated prior to reacting with the isocyanate material.

Foam density in the range of 0.02 to 0.10 g/cm³ are acceptable. Depending upon the application, foam densities above 0.10 g/cm³ may be acceptable. Generally, for insulation foams, density is typically less than 0.05 g/cm³. One skilled in the art can manipulate the total formulation for making polyurethane and polyisocyanurate foams to produce the density needed for a particular application.

From the above examples it is determined that unrefined crude glycerin is the best feedstock for use in the production of a good polyol, including fatty acid/soaps and MEFA; and that synthesis of pure glycerin/fatty acid (soap) mixtures, pure glycerin/MEFA mixtures, pure glycerin/fatty acids/MEFA, or pure glycerin/crude glycerin mixtures, approaches but does not quite reach the results using URCG. These results are evaluated primarily by hydroxyl number but also with regard to viscosity, acid number and density of a polyurethane foam produced from the polyol. It is preferred that, with unrefined crude glycerin as the alcohol reactant, the URCG should contain, inclusive of methanol and moisture, about 19 wt. % to 60 wt. % glycerin, and about 81 wt. % to 40 wt. % total fatty acids/soaps or MEFA (the term “fatty acids” or “total fatty acids” as used in the claims is intended to refer to fatty acids, soap and MEFA, any or all of which may be present when the term is used). More particularly, glycerin in the URCG is preferred in a range of about 25 wt. % to 55 wt. %, total fatty acid/soap and/or MEFA at about 45 wt. % to 75% wt. %, with MEFA about 0% to 75 wt. %. The inclusion of MEFA is shown in the above examples to reduce viscosity and can be important where lower viscosities are important. Preferred minimum content of fatty acid/soap is about 0%, 20%, 30%, or about 40%, or about 45%, or 50%, or 60%. Preferred minimum content of MEFA is about 0 wt. %, or 10%, or 20%, or 40%, or 50% or 60% or 75%. The combined percentage by weight of fatty acid/soap and MEFA will range from about 40 wt. % to 81 wt. % of the total solvent.

The above examples reflect the production of polyurethane foams from liquefied biomass-based polyols from glycerin and impurities without biomass. Polyurethanes are formed from the general reaction of an isocyanate (A-side) and an alcohol component (B-side) to form the urethane monomer that makes up the polyurethane polymer network. The isocyanate is a compound that provides the source of —NCO groups to react with functional groups from the polyol, water, and cross-linkers in the formulation.

Polyurethane foams can be produced by reacting a polyol composition with an isocyanate such as diphenylmethane diisocyanate according to known methodology. Typically, the polyol composition, a blowing agent (to form bubbles), a catalyst, and a surfactant are mixed together before adding the isocyanate. Reaction conditions, blowing agent selection, polyol selection, and isocyanate selection can all be adjusted to control the type of polyurethane that is produced (e.g., flexible, semi-rigid, or rigid polyurethane foam). Polyurethane foams produced with the polyol compositions can be used as packaging, construction and insulating materials, and can be formulated to be biodegradable. Additionally, other polyurethane forms (non-foamed) can be made with this bio-based polyol for applications such as coatings, sealants, adhesives, and elastomers.

Isocyanates which may be used in the present invention include aliphatic, cycloaliphatic, arylaliphatic and aromatic isocyanates. Examples of suitable aromatic isocyanates include the 4,4′-, 2,4′ and 2,2′-isomers of diphenylmethane diisocyanate (MDI), blends thereof and polymeric and monomeric MDI blends, toluene-2,4- and 2,6-diisocyanates (TDI),—and p-phenylenediisocyanate, chlorophenylene-2,4-diisocyanate, diphenylene-4,4′-diisocyanate, 4,4′-diisocyanate-3,3′-dimethyldiphenyl, 3-methyldiphenyl-methane-4,4′-diisocyanate and diphenyletherdiisocyanate and 2,4,6-triisocyanatotoluene and 2,4,4′-triisocyanatodiphenylether.

Mixtures of isocyanates may be used, such as the commercially available mixtures of 2,4- and 2,6-isomers of toluene diisocyanates. A crude polyisocyanate may also be used in the practice of this invention, such as crude toluene diisocyanate obtained by the phosgenation of a mixture of toluene diamine or the crude diphenylmethane diisocyanate obtained by the phosgenation of crude methylene diphenylamine. TDI/MDI blends may also be used.

Examples of aliphatic polyisocyanates include ethylene diisocyanate, 1,6-hexamethylene diisocyanate, isophorone diisocyanate, cyclohexane 1,4-diisocyanate, 4,4′-dicyclohexylmethane diisocyanate, saturated analogues of the above mentioned aromatic isocyanates and mixtures thereof. For the production of flexible foams, polyisocyanates that are particularly useful include toluene-2,4- and 2,6-diisocyanates or MDI or combinations of TDI/MDI or prepolymers made therefrom. Isocyanates useful herein typically contain at least two isocyanate groups per molecule. The Dow Chemical Company offers a broad range of isocyanates which include ISONATE® MDI (methylene diphenyl diisocyanate) and PAPI® polymeric MDI for polyurethane processing and solution applications in coatings, adhesives, sealants and elastomers. For example, PAPI® 27 is a polymeric MDI with a 31.4% weight NCO, 134 isocyanate equivalent weight, >204° C. flash point, and 180 cP viscosity at 25° C. In polyurethane chemistry, the isocyanate index is the amount of isocyanate used relative to the theoretical equivalent amount, i.e., 100 times the ratio of NCO groups to reactive hydrogen of the polyol composition and the reaction mixture. The isocyanate index typically ranges from about 5 to about 150, more particularly from about 10 to about 110; rigid foams typically have an index of 105-110. Flexible foams 105-115, occasionally 85-110.

One or more crosslinkers may be present in the foam formulation. If used, suitable amounts of crosslinkers are from about 0.1 to about 1 part by weight, especially from about 0.25 to about 0.5 part by weight, per 100 parts by weight of polyols. As used herein, “crosslinkers” are materials having three or more isocyanate-reactive groups per molecule and an equivalent weight per isocyanate-reactive group of less than 400. In accordance with some aspects, crosslinkers contain from 3-8, especially from 3-4 hydroxyl, primary amine or secondary amine groups per molecule and have an equivalent weight of from 30 to about 200, especially from 50-125. Examples of suitable crosslinkers include diethanol amine, monoethanol amine, triethanol amine, mono- di- or tri(isopropanol)amine, glycerine, trimethylol propane, pentaerythritol, sorbitol and the like.

It is also possible to use one or more chain extenders in the foam formulation. As used herein, a chain extender is a material having two isocyanate-reactive groups per molecule and an equivalent weight per isocyanate-reactive group of less than 400, especially from 31-125. The isocyanate reactive groups are preferably hydroxyl, primary aliphatic or aromatic amine or secondary aliphatic or aromatic amine groups. Representative chain extenders include amines, ethylene glycol, diethylene glycol, 1,2-propylene glycol, dipropylene glycol, tripropylene glycol, ethylene diamine, phenylene diamine, bis(3-chloro-4-aminophenyl) methane and 2,4-diamino-3,5-diethyl toluene. If used, chain extenders are typically present in an amount from about 1 to about 50, especially about 3 to about 25 parts by weight per 100 parts by weight high equivalent weight polyol, the contents of which are hereby incorporated by reference. The use of such crosslinkers and chain extenders is known in the art as disclosed in U.S. Pat. No. 4,863,979 and EP Publication 0 549 120, the contents of which are hereby incorporated by reference.

Any commercial blowing agent or method to cause expansion of the material can be used in forming the foam of the present invention. Examples of blowing agents that may be used include, but are not limited to, water, carbon dioxide, and pentane and pentane isomers, as well as hydroflorocarbons. The blowing agent functions to expand the product and turn it into a foam. Water is a particularly useful blowing agent for formulations of the present invention. In formulations useful for preparing the polyurethane foams of the present invention, water may be present at a concentration of from about 0.75 to 6 weight percentage of biopolyols. In accordance with certain embodiments, water may be present at from about 1.0 to 6.0 weight percentage of B-side component. In accordance with other embodiments, water may be present at from about 2.0 to 4.0 weight percentage of B-side component.

While some polyurethane foam formulations may include water as the only blowing agent, it is also contemplated that the present invention includes formulations having minor amounts of auxiliary blowing agents as well. When an auxiliary blowing agent is used, it may be present between about 0.01 to 10 weight percentage of the B-side component. More particularly, the auxiliary blowing agent may be present between about 0.1 to 5 weight percentage. For example, both water and one or more of the following materials could be used as blowing agents for the formulations of the present invention: HCFC-22, HFC-134a, HCFC-142b, HFC-245fa, dichloroethylene, hydrocarbons such as n-pentane, isopentane, cyclopentane and the like. Small amounts of such auxiliary blowing agents when used with the composition of the present invention may result in higher insulation values. In accordance with other aspects, a blowing agent or blend of blowing agents can be used that does not contain water. The B-side component may include some metal salt catalyst. The metal salt catalysts, along with the excess heat of reaction, causes the residual isocyanate to react with itself to form very stable isocyanurate functionality. The metal salt catalysts are alkali metal salts of organic acids, more particularly sodium or potassium salts. Metal salt catalyst may be present between about 0.05 to 10 weight percent. Examples of commercially available metal salt catalyst suitable for the present invention include DABCO® K-15 and Polycat® 46 from Air Products, and the like. Other common catalysts used for flexible foam formulation include Stannous octoate and Dibutyltin dilaurate—both tin-based.

In addition to the above referenced components, the B-side component may also include additional catalysts, surfactants, flame retardants, colorants or other additives such as would be known to those of skill in the art. The B-side component may include at least one amine catalyst. Commercially available amine catalysts suitable for the present invention include Polycat® 5, Polycat® 8, Polycat® 11, DABCO® 33 LV, DABCO® BL-17 and DABCO® BL-11 from Air Products. Other amine catalysts can also be used. In accordance with certain embodiments, the amine catalyst may be present in an amount between about 0.1 and about 4.0 weight percentage of the B-side component.

Surfactants may be present in an amount between about 0.25 and about 3.0 weight percentage of the B-side component. These surfactants typically take the form of polydimethylsiloxane-polyoxyalkylene block copolymers, silicone oils, nonylphenol ethoxylates, and other organic compounds. Commercially available surfactants suitable for the present invention include DABCO® DC5357, DABCO® DC2585, DABCO® DC193, DABCO® DC4053, DABCO® DC5098 from Air Products, and the like.

Additionally, flame retardants can be used in the B-side component of the present invention. In accordance with some embodiments, between about 5.0 and about 15 weight percentage flame retardant may be used. Frequently used flame retardants include tris(chloroethyl) phosphate, tris(chloroisopropyl) phosphate and tris(dichloroisopropyl) phosphate. U.S. Pat. No. 3,830,890 describes tetra esters of 2-butene-1,4-diphosphonic acid as flame retardants for polyurethane foams. U.S. Pat. No. 4,067,931 describes tetraalkyl esters of polyoxymethylenediphosphonic acid as flame retardants for polyurethane foams. Other flame retardant chemicals used in polyurethane foams include triphenyl phosphate, chloroalkyl phosphate, aryl phosphates and other organic phosphate esters as well as other materials known to one skilled in the art. The A:B volume ratio typically is between about 1:1 and about 3:1. More particularly, the A:B volume ratio typically is between about 1.2:1 and about 2.5:1. Even more particularly, the A:B volume ratio typically is between about 1.25:1 and about 2.0:1. In accordance with particular embodiments, the A:B volume ratio may be about 1.5:1.

Processes for producing polyurethane products from the polyols described herein are not particularly limited, and any of the processes known in the art can be used. Typically, the components of the polyurethane-forming reaction mixture may be mixed together in any convenient manner, for example, by using any of the mixing equipment described in the prior art for the purpose such as described in “Polyurethane Handbook,” by G. Oertel, Hanser publisher. The polyurethane products may be produced either continuously or discontinuously, by injection, pouring, spraying, casting, calendering, etc. Polyurethane products may be produced under free rise or molded conditions, with or without release agents, etc. Additionally, polyurethane products may be produced that are not foamed.

Rigid foams may be produced using the known 1-shot prepolymer or semi-prepolymer techniques, together with conventional mixing methods including impingement mixing. The rigid foam may also be produced in the form of slabstock, bunstock, moldings, cavity filing, sprayed foam, frothed foam or laminates with other materials such as paper, metal, plastics or wood-board. Flexible foams may be either free rise or molded while microcellular elastomers are usually molded.

Polyurethane foam produced in accordance with certain aspects of the present invention can be applied to a surface or substrate by reacting the A-side component and the B-side component to form a polyurethane reaction product and applying the polyurethane reaction product to the surface or substrate. Examples of surfaces to which the polyurethane foam can be applied include, without limitation, roofs, structural walls, exterior surfaces, interior surfaces, storage tanks, insulated cavities, and process vessels.

Several important properties of the biopolyol dictate polyurethane formulation. These physicochemical properties include acid number, hydroxyl number and viscosity. Acid and hydroxyl numbers are needed to calculate the amount of isocyanate needed for urethane reaction. Acid number (mg KOH/g) is defined as the amount in milligrams of potassium hydroxide required to neutralize the acid present in one gram of a polyol sample (usually present as acid residuals in the polyol). Typical values of acid number in commercially-available polyols are less than 10 mg KOH/g sample, often preferably less than 5 mg KOH/g. Low acidity in some polyols can be important because high acid number polyols tend to neutralize the urethane formulation catalysts and react with isocyanate to compete with hydroxyls in the urethane formation. In accordance with certain aspects, the measured acid numbers of the produced biopolyols range from about 0.1 to 2.0 mg KOH/g, more preferably less than about 10, or less than about 7.

Hydroxyl Number is an index of the amount of reactive hydroxyl groups available for reaction. This value is determined using a wet analytical method and is reported as mg KOH/g sample (amount in milligrams of KOH equivalent to the hydroxyl groups found in a gram of a polyol sample). Rigid foam formulation usually requires higher hydroxyl number in the range of >200 mg KOH/g. Hydroxyl number analyses of particular examples of liquefied biopolyols showed values in the 200˜800 mg KOH/g range, more particularly from about 400-750 mg KOH/g, which is better suited for rigid foam formulation.

The polyols produced according to the invention and having hydroxyl numbers not greater than about 1100 were found to produce good, rigid polyurethane foams. To produce flexible foams, or semi-flexible foams, polyols of lower effective hydroxyl number must be employed. For this purpose polyols produced from unrefined crude glycerin, and also from pure glycerin in a glycerin composition with soaps/fatty acids and optionally MEFA, can be blended with petroleum-based polyols having lower hydroxyl numbers. Typically petroleum-based polyols have hydroxyl number in the range of about 20 to 600 mg KOH/g. For flexible foams petroleum-based polyols toward the lower end of this range are blended with polyols produced according to the invention, whether from liquefaction of biomass or without biomass. Good quality flexible foams have been produced using 40% biopolyols made according to the invention, and also up to 50%, of the blend being expressed as weight percent of the polyol blend.

Viscosity is considered an important physical property of the polyol. It indicates the degree of oligomerization of the reactants (cellulose and glycerol, among others). In accordance with certain aspects, the viscosity of the polyol composition may fall within a range from about 200-50,000 cP, more preferably about 10,000 cP or less. The dynamic viscosities (centipoise, cP measured at 20° C. or 25° C.) of the samples may be determined using a commercially-available rheometer, example—Model RS100 Rheometer (Haake-Thermoelectron, Newington, N.H.). Higher average molecular weights of the biopolyol can have a significant impact in later PU formulations.

The biopolyols in accordance with one particular example were synthesized using soybean straw as the biomass source, crude glycerin as solvent, and sulfuric acid as catalyst. Soybean straw and sulfuric acid were added at mass ratios of 0.5-15% (based on crude glycerin) and 2-8% (based on crude glycerin), respectively. About 200 grams of crude glycerin (as produced as a byproduct of biodiesel production with only minimal refinement to remove floating soaps, etc) was mixed with soybean straw and sulfuric acid in a 500-mL round-bottom three-necked flask. The mixture was heated to a temperature range of 190° C.-200° C. for 60-90 minutes using the heater-condenser setup described above. The synthesized biopolyols were characterized for PU foam formulation. Polyols with workable viscosity (−200-10,000 cP) and acid numbers less than 5 mg KOH/g were used for foaming. A modified foaming procedure was used in preparing a PU foam example. About 60 g of liquefied biopolyol, 0.756 g Polycat® 5, 0.504 g Polycat® 8, 1.5 g DABCO® DC5357 and 1-6% water as blowing agent were added by weighing into a 400-mL disposable paper drinking cup and mixed at high speed with an electric mixer for 15 seconds. The mixture was allowed to degas for 120 seconds after which the A-side component isocyanate PAPI® 27 was rapidly added while continuously stirring for another 1015 seconds at the same speed. The foam mixture was immediately poured into a wooden mold (11.4 cm×11.4×21.6 cm) lined with aluminum foil and was allowed to rise and set at ambient conditions (23° C.)

Molds in different shapes and dimensions can be used depending on the final use of the PU foams. The proposed bio-based PU can be potentially used for foams, coatings, adhesives, sealants, elastomers and any other typical use for PU. PU foams can be used for insulation, packaging, construction, automotive, furniture, bedding, agricultural film, and door panels, etc. Foams produced in accordance with certain aspects of the present invention are suitable for use in those applications typically used for polyurethane foam. For example, rigid foams can be used in the construction industry and for insulation for appliances, while flexible foams and elastomers can be used in applications such as furniture, mattresses, shoe soles, automobile seats, sun visors, steering wheels, arm rests, door panels, noise insulation parts and dash boards. Foams can be prepared and utilized in various forms, such as molded packing foam, molded insulation boarding, spray-on insulation and spray-in protective foam for packaging. Foams can be prepared as planks or buns.

The PU foam is expected to be biodegradable and would be particularly useful in the production of loose-fill packing material such as packaging peanuts. PU foams produced in accordance with certain aspects of the present invention may be water resistant. The PU foams produced in accordance with certain aspects are considered to be closed cell which means that each cell which makes up the foam structure is completely closed-off to surrounding cells. This prevents water or moisture from entering the cells for short periods of time (up to several months). Long-term exposure to water may soften the polyurethane structure due to its partly biodegradable property.

In the claims, as noted above, “fatty acid” refers to fatty acid, soap and/or MEFA, any or all of which can be included when the term is used. “Soap” indicates soap, salt or MEFA. MEFA refers to methyl esters of fatty acid, i.e. biodiesel. “Alcohol reactant” means glycerin with contaminants (“fatty acid” as defined), whether occurring in feedstock UCRG or added to pure glycerin.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A method for producing a polyol, comprising:
providing an alcohol reactant comprising unrefined crude glycerine, the unrefined crude glycerine being a by-product of a transesterification process of non-petroleum oil sources for producing biodiesel fuel, the unrefined crude glycerine including at least about 30% fatty acid by weight of the unrefined crude glycerine, and
heating the alcohol reactant to a sufficient temperature for a sufficient time to produce a liquid polyol wherein said liquid polyol is capable of being reacted with an isocyanate to produce a useful polyurethane.

2. A method in accordance with claim 1, wherein the alcohol reactant comprises at least 90% unrefined crude glycerine by weight based on the total alcohol reactant weight.

3. A method in accordance with claim 1, wherein acid is added to the unrefined crude glycerin to produce a pH of about 6 to 8.

4. A method in accordance with claim 1, wherein acid is added to the unrefined crude glycerin to produce a pH of about 6.5 to 7.5.

5. A method in accordance with claim 1, wherein the unrefined crude glycerine includes at least about 40% fatty acid by weight.

6. A method in accordance with claim 1, wherein the unrefined crude glycerine includes at least about 45% fatty acid by weight.

7. A method in accordance with claim 1, wherein the unrefined crude glycerine includes at least about 50% fatty acid by weight.

8. A method in accordance with claim 1, wherein the unrefined crude glycerine includes at least about 60% fatty acid by weight.

9. A method in accordance with claim 1, wherein the unrefined crude glycerine includes about 45% to 60% fatty acid by weight.

10. A method in accordance with claim 9, wherein the fatty acid fraction includes methyl ester of fatty acid.

11. A method in accordance with claim 1, wherein the unrefined crude glycerine includes at least about 5% methyl ester of fatty acid by weight of the unrefined crude glycerine.

12. A method in accordance with claim 1, wherein the unrefined crude glycerine includes at least about 10% methyl ester of fatty acid by weight of unrefined crude glycerine.

13. A method in accordance with claim 1, wherein the step of heating the alcohol reactant comprises heating to a temperature of at least about 150° C.

14. A method in accordance with claim 1, wherein the step of heating the alcohol reactant comprises heating to a temperature of at least about 160° C.

15. A method in accordance with claim 1, wherein the step of heating the alcohol reactant comprises heating to a temperature of at least about 180° C.

16. A method in accordance with claim 1, wherein the step of heating the alcohol reactant comprises heating to a temperature of at least about 190° C.

17. A polyol composition produced in accordance with the method of claim 1, wherein said polyol has a viscosity falling within a range from about 100 to 50,000 cP at 25° C.

18. The polyol composition of claim 17, wherein the polyol has a hydroxyl number of between about 200-800 mg KOH/g.

19. The polyol composition of claim 17, wherein the polyol has a hydroxyl number of between about 150-750 mg KOH/g.

20. The polyol composition of claim 17, wherein the polyol has an acid number of between about 0.5-5 mg KOH/g.

21. The polyol composition of claim 17, wherein the polyol has an acid number less than about 5 mg KOH/g.

22. A method in accordance with claim 1, wherein the alcohol reactant consists essentially of unrefined crude glycerin.

23. A method in accordance with claim 1,
including the step of adding MEFA into the alcohol reactant, to a range up to about 25% by weight of total alcohol reactant including the MEFA.

24. A method in accordance with claim 23, wherein the MEFA is added to a range up to about 15% by weight of total alcohol reactant including the MEFA.

25. A method in accordance with claim 1, including the step of adding MEFA into the alcohol reactant, to a range up to about 60% by weight of total alcohol reactant including the MEFA.

26. A method in accordance with claim 1, wherein the alcohol reactant is heated as part of an admixture with lignocellulosic biomass.

27. A method in accordance with claim 26, wherein the ratio of biomass to alcohol reactant is about 0.05% to 20%, wherein the ratio is expressed as a percentage equivalent to the mass of biomass divided by the mass of solvent.

28. A method for producing a polyol, comprising:
providing an alcohol reactant comprising a glycerine composition with at least about 30% fatty acid by weight of the glycerine composition, and
heating the alcohol reactant to a sufficient temperature
and for a sufficient time to produce a liquid polyol wherein said liquid polyol is capable of being reacted with an isocyanate to produce a useful polyurethane.

29. A method in accordance with claim 28, wherein the composition includes at least about 40% fatty acid by weight.

30. A method in accordance with claim 28, wherein the glycerine composition includes at least about 50% fatty acid by weight.

31. A method in accordance with claim 28, wherein the unrefined crude glycerine includes at least about 60% fatty acid by weight.

32. A method in accordance with claim 28, wherein the step of heating the alcohol reactant comprises heating to a temperature of at least about 160° C.

33. A method in accordance with claim 28, wherein the step of heating the alcohol reactant comprises heating to a temperature of at least about 185° C.

34. A method in accordance with claim 28, wherein the alcohol reactant is heated as part of an admixture with lignocellulosic biomass.

35. A method in accordance with claim 28, including the step of adding MEFA into the alcohol reactant, to a range up to about 25% by weight of total alcohol reactant.

36. (canceled)

37. A method in accordance with claim 36, wherein the ratio of biomass to alcohol reactant is about 0.05% to 20%, wherein the ratio is expressed as a percentage equivalent to the mass of biomass divided by the mass of solvent.

38. A polyurethane produced with a polyol made according to the method of claim 28, with the further step of reacting the polyol with an isocyanate.
